SEQUENCE LISTING (16 sequences)

SEQ ID NO: 1 - DNA, 22 bp, Artificial Sequence
  Genomic sequence of C. suis
gcttcgaatggccgcataaagg

SEQ ID NO: 2 - DNA, 22 bp, Artificial Sequence
  Reverse primer
cctttatgcggccattcgaagc

SEQ ID NO: 3 - DNA, 20 bp, Artificial Sequence
  Reverse primer
cctttatgcggccattcgaa

SEQ ID NO: 4 - DNA, 20 bp, Artificial Sequence
  Reverse primer
tttatgcggccattcgaagc

SEQ ID NO: 5 - DNA, 15 bp, Artificial Sequence
  Reverse primer
ttatgcggccattcg

SEQ ID NO: 6 - DNA, 22 bp, Artificial Sequence
  Reverse primer
tatgcggccattcgaagcagcc

SEQ ID NO: 7 - DNA, 21 bp, Artificial Sequence
  Forward primer
gatcattcacacgtggccctt

SEQ ID NO: 8 - DNA, 19 bp, Artificial Sequence
  Forward primer
tcattcacacgtggccctt

SEQ ID NO: 9 - DNA, 19 bp, Artificial Sequence
  Forward primer
gatcattcacacgtggccc

SEQ ID NO: 10 - DNA, 19 bp, Artificial Sequence
  Forward primer
atcattcacacgtggccct

SEQ ID NO: 11 - DNA, 23 bp, Artificial Sequence
  Forward primer
gatcattcacacgtggcccttga

SEQ ID NO: 12 - DNA, 21 bp, Artificial Sequence
  Forward primer
tcattcacacgtggcccttga

SEQ ID NO: 13 - DNA, 19 bp, Artificial Sequence
  Probe
gcgttcgggggaagctgtg

SEQ ID NO: 14 - DNA, 21 bp, Artificial Sequence
  Probe
gagcgttcgggggaagctgtg

SEQ ID NO: 15 - DNA, 20 bp, Artificial Sequence
  Probe
gcgttcgggggaagctgtgt

SEQ ID NO: 16 - DNA, 22 bp, Artificial Sequence
  Probe
gagcgttcgggggaagctgtgt

(19) United States
(12) Patent Application Publication
(10) Pub. No.: US 2024/0247321 A1
(43) Pub. Date: Jul. 25, 2024

(54) METHOD FOR DETECTING CYSTOISOSPORA SUIS

(71) Applicant: Ceva Sante Animale, Libourne (FR)

(72) Inventors: Zalan Hommonnay, Budapest (HU); Itsvan Kiss, Budapest (HU)

(21) Appl. No.: 18/030,580

(22) PCT Filed: Oct. 6, 2021
(86) PCT No.: PCT/EP2021/077568

§ 371 Date: Apr. 6, 2023

(30) Foreign Application Priority Data
Oct. 7, 2020 (EP) 20306162.7

(51) Int. Cl. C12Q 1/6893

(52) U.S. Cl. CPC C12Q 1/6893

(57) ABSTRACT

The present invention relates to a method for detecting Cystoisospora suis in a sample. The invention also relates to kits and materials, such as primers or probes, which can be used for such detection. The invention may be used in any sample, such as a biological sample, and allows specific detection of Cystoisospora suis.

The present invention relates to a method for detecting Cystoisospora suis in a sample. The invention also relates to kits and materials, such as primers or probes, which can be used for such detection. The invention may be used in any sample, such as a biological sample, and allows specific detection of Cystoisospora suis.

### BACKGROUND

Cystoisospora suis (C. suis) is an important coccidian protozoan parasite causing severe diarrhoea in piglets. Detecting C. suis in samples can thus allow proper treatment to avoid spreading and/or disease progression.

Enumeration of the oocysts by fluorescent microscopy and flotation techniques can be used to identify the agent in the faeces. Such methods, however, are not practical and are long lasting.

A detection assay for Cystoisospora has been reported by Samarasinghe et al. 2008 (Experimental Parasitology 118: 592-595.). The method, however, is not species-specific but rather reacts with various species of the Cystoisospora genus.

There is a need in the art for methods allowing more specific detection of C. suis.

### SUMMARY OF THE INVENTION

The present invention relates to a novel method and novel tools (reagents, kits) for detecting Cystoisospora suis in a sample. The method uses amplification, such as PCR, and can be implemented with any sample, such as biological samples (or diluents/derivatives thereof), such as fluids, stool, faeces, tissues, etc. The inventors designed reverse primers and probes allowing amplification/detection of a particular target sequence in the Internal Transcribed Spacer (“ITS”) region of the genome, and showed such method and reagents allow more specific and efficient detection of C. suis. In particular, the invention shows that targeting a genomic sequence within the 16S-23S rRNA ITS region of the mitochondrial genome of C. suis allows efficient and specific detection. Thermal profile of the assay was optimized by gradient real-time PCR. Moreover, parameters of mechanical disruption and enzymatic lysis of faecal samples were also optimized for DNA extraction. A full method including DNA-extraction and real-time PCR assay, applicable to 96-well plates, was also designed, allowing high throughput and efficient detection of C. suis, and showing no cross reactivity with other Cystoisospora species C. ochioensis and C. belli.

### DETAILED DESCRIPTION OF THE INVENTION

The present invention relates to a novel method and novel tools (reagents, kits) for detecting (the presence, or absence, or amount of) Cystoisospora suis in a sample. The method is based, inter alia, on the amplification of a particular sequence of C. suis genome, allowing more specific (e.g., no cross reactivity with C. ochioensis or C. belli) and efficient detection of C. suis. The invention also stems from the design of improved sample treatment, allowing efficient detection of C. suis.

The invention thus relates to a method of detecting C. suis in a sample, comprising a step of amplifying a target sequence in the genome of C. suis. The invention is typically implemented in vitro.

The method may further comprise a step of detecting presence (or absence) of an amplicon using a specific probe.

The method may further comprise a step of treating the sample prior to amplification.

The invention also relates to particular primers and probes, as well as to kits comprising the same.

The invention also relates to a method for detecting C. suis infection in a sample, using the above method, reagents and/or kits.

The invention also relates to a method for treating C. suis infection in a mammal, particularly a piglet, comprising (i) detecting or confirming the infection using a method, kit or reagent as defined above and (ii) treating the infection.

### Targeted Genomic Sequence and Primers

The invention shows that targeting a genomic sequence within the 16S-23S rRNA ITS region of the mitochondrial genome of C. suis allows efficient and more specific detection. More specifically, the invention is based, inter alia, on the amplification of a sequence of less than 400 bp within the 16S-23S rRNA ITS region of the mitochondrial genome of C. suis. More preferably, the amplified sequence contains more than 50 bp and less than 250 bp, even more preferably more than 50 bp and less than 200 bp.

In a particular embodiment, amplification is performed with a pair of primers comprising a forward and a reverse primer, wherein the reverse primer hybridizes specifically to (i.e., is perfectly complementary to) all or a portion of the following genomic sequence: 5′-GCTTCGAATGGCCGCATAAAGG-3′ (SEQ ID NO: 1). The primer may be fully overlapping to the above sequence (in all or in part), or to at least 60% of said target genomic sequence, even more preferably at least 70%, or 80% at least of said sequence (in such a case, the remaining portion of the primer is complementary to a sequence flanking SEQ ID NO: 1 in the genome of C. suis).

Preferred primers typically comprise a single-strand nucleic acid, with a length advantageously between 5 and 50 bases, preferably between 5 and 30 bases, even more preferably 10-30, or 15-25.

As indicated above, the reverse primer may be centred on the above target sequence, or partially overlapping therewith, by at least 60%, more preferably at least 70%, or at least 80%. An overlap of 80% indicates that the primer should overlap to at least 18 consecutive bases of SEQ ID NO: 1.

Specific and preferred examples of suitable reverse primers for use in the invention are disclosed below:

(SEQ ID NO: 2)
CCTTTATGCGGCCATTCGAAGC
(SEQ ID NO: 3)
CCTTTATGCGGCCATTCGAA
(SEQ ID NO: 4)
TTTATGCGGCCATTCGAAGC
(SEQ ID NO: 5)
TTATGCGGCCATTCG
(SEQ ID NO: 6)
TATGCGGCCATTCGAAGCAGCC

A further object of the invention relates to the use of a nucleic primer selected from anyone of SEQ ID NOs: 2-6, in a method of detecting C. suis.

A further object of the invention relates to the use of a nucleic primer selected from anyone of SEQ ID NOs: 2-6, for amplifying C. suis genomic sequence in a sample.

A further object of the invention relates to a method for detecting C. suis in a sample, comprising a step of nucleic acid amplification in said sample with a nucleic primer selected from anyone of SEQ ID NOs: 2-6.

The forward primer may be any primer allowing, in combination with the reverse primer, amplification of a C. suis genomic sequence of between 50 and 400 bp, preferably between 50 and 250 bp, even more preferably between 50 and 200 bp.

Examples of preferred forward primers suitable for use in the invention are provided below:

(SEQ ID NO: 7)
GATCATTCACACGTGGCCCTT
(SEQ ID NO: 8)
TCATTCACACGTGGCCCTT
(SEQ ID NO: 9)
GATCATTCACACGTGGCCC
(SEQ ID NO: 10)
ATCATTCACACGTGGCCCT
(SEQ ID NO: 11)
GATCATTCACACGTGGCCCTTGA
(SEQ ID NO: 12)
TCATTCACACGTGGCCCTTGA

An object of the invention thus relates to a method for detecting C. suis in a sample, comprising a step of nucleic acid amplification in said sample with a pair of a forward and a reverse nucleic primers, the reverse primers being selected from anyone of SEQ ID NOs: 2-6 and the forward primer being selected from anyone of SEQ ID NOs: 7-12. Any combination of said primers is suitable, as shown in the examples.

A further object of the invention relates to the use of a pair of nucleic primers for amplifying C. suis genomic sequence in a sample, the pair comprising a reverse primer selected from anyone of SEQ ID NOs: 2-6 and a forward primer selected from anyone of SEQ ID NOs: 7-12.

### Method of Amplification

Various techniques for amplifying a nucleic acid in a sample can be used in the present invention. Amplification can be performed according to various methods known per se to the person skilled in the art, such as PCR (Polymerase Chain Reaction), LCR, transcription mediated amplification (TMA), strand displacement amplification (SDA), NASBA, the use of allele specific oligonucleotides (ASO), allele specific amplification, RNAseq. Detection can also be made using, e.g., Southern blot, single-strand conformation analysis (SSCA), in situ hybridisation (e.g., FISH), gel migration, heteroduplex analysis, NextGen sequencing, etc.

In a preferred embodiment, amplification is performed by PCR, such as for example RT-PCR, quantitative PCR (qPCR) or ligation-PCR. More preferably, the method uses qPCR.

According to a preferred embodiment, the method comprises determining the presence or absence or (relative) amount of C. suis by qPCR amplification.

The method is typically performed in vitro, in a test sample. The method may be conducted on individual samples, or on multisupport such as plates, allowing several test to be conducted essentially simultaneously.

### Probes

In a particular embodiment, amplification is performed in the presence of a labelled probe, allowing detection of the presence of an amplicon. The probe is a single stranded oligonucleotide, of typically 5-30 nt long, which specifically hybridizes to a portion of the amplified sequence. The probe is typically labelled, such as with fluorescent label (e.g., SYBR, FAM, BHQ1, etc). In a preferred embodiment, the probe is or comprises all or part of SEQ ID NO: 13:

(SEQ ID NO: 13)
GCGTTCGGGGGAAGCTGTG

Such probe, in combination with the above primers, allows efficient and more specific detection of C. suis, especially by qPCR.

Other alternative probes may be selected from the following sequences:

(SEQ ID NO: 14)
GAGCGTTCGGGGGAAGCTGTG
(SEQ ID NO: 15)
GCGTTCGGGGGAAGCTGTGT
(SEQ ID NO: 16)
GAGCGTTCGGGGGAAGCTGTGT

### Samples and Treatment

The method of the invention is applicable to any sample, such as any biological sample. Because C. suis is responsible for severe infections in mammals such as pigs (swine), the sample is typically a biological sample obtained (or derived) from a tested mammal, such as a pig, suspected of C. suis infection. Specific examples of such biological samples include any tissue, organ or biological fluid that can include nucleic acids, such as faeces, stool, blood, urine, saliva, etc. Advantageously, the method can be implemented with biological samples such as faeces. The invention may also be applied to any sample (natural, soil, etc) for which detection of C. suis is needed or of use.

The sample can be pre-treated to facilitate/normalize access to the target molecules, for example by lysis (mechanical, chemical, enzymatic, etc.), purification, centrifugation, separation, etc. The sample can also be labelled to facilitate the determination of the presence of target molecules (biotin, fluorescent, radioactive, luminescent, chemical or enzymatic labelling, etc.). The nucleic acids of the sample can in addition be separated, treated, enriched, purified, fragmented, etc. Typically, the sample is treated for DNA extraction.

In a particular embodiment, the sample is faeces from a mammal, more particularly pig faeces (or stool)

In a further particular embodiment, the sample is faeces which is pretreated for DNA extraction. Preferably, DNA extraction comprises a lysis and, optionally, a centrifugation and/or proteinase treatment.

In a particular embodiment, the method comprises:
a) providing one or several biological samples comprising DNA extracted from stool or faeces of a mammal to be tested, and
b) detecting the presence of C. suis in said sample according to a method as define above.

### Kits

Another object of the present application relates to a kit comprising a primer as defined above.

The kit typically comprises a pair of primers as defined above, which may be in a same or separate containers. The kit also typically comprises a probe as defined above, and/or reagents for performing an amplification reaction.

A particular object of the invention resides in a kit comprising a nucleic acid primer selected from anyone of SEQ ID NOs: 2-6 and one or more reagent(s) for performing an amplification.

In a particular embodiment, the kit further comprises a primer selected from anyone of SEQ ID NOs: 7-12.

In a further particular embodiment, the kit further comprises a labeled probe selected from anyone of SEQ ID NOs: 13-16.

Another object of the invention relates to the use of a primer or kit such as defined above for the detection of C. suis in a sample, or for the detection of C. suis infection in a mammalian subject, preferably a pig.

Further aspects and advantages of the present invention will appear upon consideration of the following examples, which must be regarded as illustrative and non-restrictive.

### EXAMPLES

### Example 1. DNA Extraction from Fecal Samples (Few Samples)

For DNA extraction, QIAGEN's QIAamp® PowerFecal® DNA Kit was used, with modifications on the manufacturer's recommended protocol as disclosed below. Centrifugation steps were performed at room temperature (15-25° C.).

0.1 g of stool was added deep into the Bead Tube and the PowerBead Solution was added. 80 μl of Solution C1 was then added and the tube was inverted several times and then heated at 65° C. for 20 min. The tubes were secured horizontally and then centrifuged at 13,000×g for 1 min. 600 μl of the supernatant was transferred to a clean 2 ml Collection Tube, to which 300 μl of Solution C2 was added and vortexed briefly to mix. Incubation was performed at 2-8° C. for 10 min, and the tubes were then centrifuged at 13,000×g for 1 min. The supernatant was transferred to a clean 2 ml Collection Tube, to which 250 μl of Solution C3 was added and vortexed briefly. Incubation was performed at 2-8° C. for 5 min, and the tubes were then centrifuged at 13,000×g for 1 min. The supernatant was transferred to a clean 2 ml Collection Tube, to which 1200 μl of Solution C4 was added and vortexed. 650 μl of supernatant was then loaded onto an MB Spin Column, and centrifuged at 13,000×g for 1 min. The flow-through was discarded and the step was repeated twice (altogether three loads). 500 μl of Solution C5 was added and centrifuged for 1 min at 13,000×g. The flow-through was discarded and the rest was centrifuged again for 2 min at 13,000×g. The MB Spin Column was placed in a clean 2 ml Collection Tube and 100 μl of Solution C6 was added to the center of the filter membrane, followed by centrifugation at 13,000×g for 1 min and discard of the MB Spin Column.

The tube contains the extracted DNA under conditions suitable for amplification.

### Example 2. DNA Extraction from in 96-Well Plate Design

For DNA extraction, QIAGEN's QIAamp® 96 PowerFecal QIAcube® HT Kit (with QIAcube® HT nucleic acid extraction device) was used with modifications on the manufacturer's recommended protocol as disclosed below.

Up to 100 mg of each stool sample was paced into a Pathogen Lysis Tube L, 1000 μl of prewarmed Buffer PW1 was added to each sample, which were then homogenized in TissueLyser II at 20 Hz for 20 min. the tubes were centrifuged at full speed for 1 min to pellet stool particles. Pipet approximately 400 μl supernatant from into a new 1.5 ml centrifuge tube. 150 μl Buffer C3 were added to the supernatants and mixed thoroughly. Incubation was maintained for 5-10 min at 4° C., followed by centrifugation at full speed for 2 min. 20 μl Proteinase K was added to each wells of a new S-Block well, and 300 μl of each supernatant from preceding step were added to these wells and mixed, and incubated for 10 min at room temperature (15-25° C.). Subsequently, DNA extraction was completed with “QIAamp DNA Protocol on the QIAcube HT”, following the instructions of the software.

At the end of the process, 100 μl extracted was obtained in each Elution Microtube, which is in condition for amplification.

### Example 3. Detection of C. suis by Amplification

Detection was performed by qPCR. The following oligonucleotides were used:

Forward primer:
SEQ ID NO: 7
GATCATTCACACGTGGCCCTT;
Reverse primer:
SEQ ID NO; 2
CCTTTATGCGGCCATTCGAAGC;
Probe:
SEQ ID NO: 13
FAM-GCGTTCGGGGGAAGCTGTG-BHQ1;

The following reaction mix was used (based on QIAGEN's QuantiNova Probe RT-PCR kit). Other high-quality mastermixes may be applicable.
3.4 μl mol. biol. grade H₂O
10 μl QuantiNova MasterMix (2×)
0.1 μl ROX*
1 μl forw. primer (10 μM)
1 μl rev. primer (10 μM)
0.5 μl probe (10 μM)
4 μl extracted DNA
final vol.: 20 μl

### Thermal Profile:

95° C. 10 min
40 cycles of 95° C. 10 sec
   60° C. 40 sec—with data acquisition on FAM (green) channel

Samples resulting in detectable Ct during the first 40 cycles should be considered as positive.

The results show that C. suis can be efficiently detected from biological sample using such protocol. The assay is found to be specific for Cystoisospora suis, and not specific for C. ochioensis and C. belli.

Similar results can be obtained using the following combinations of oligonucleotides:

| | combiA | combiB | combiC | combiD |
|---|---|---|---|---|
| Forward | SEQ ID | SEQ ID | SEQ ID | SEQ ID |
| primer | NO: 7 | NO: 8 | NO: 9 | NO: 10 |
| Reverse | SEQ ID | SEQ ID | SEQ ID | SEQ ID |
| primer | NO: 3 | NO: 2 | NO: 4 | NO: 5 |
| Probe | SEQ ID | SEQ ID | SEQ ID | SEQ ID |
| | NO: 13 | NO: 13 | NO: 14 | NO: 15 |

The ASCII text file “Sequence.txt” created on Oct. 31, 2023, having the size of 4 KB, is incorporated by reference into the specification.

What is claimed is:

1. A method for detecting C. suis in a sample in vitro, comprising a step of amplification of a target sequence of less 400 bp within the 1 6S-23S rRNA ITS region of the mitochondrial genome of C. suis.

2. The method of claim 1, wherein amplification is performed with a pair of primers comprising a forward and a reverse primer, wherein the reverse primer hybridizes specifically to all or a portion of the following genomic sequence: 5′-GCTTCGAATGGCCGCATAAAGG-3′ (SEQ ID NO: 1).

3. The method of claim 2, wherein the reverse primer is fully overlapping to SEQ ID NO: 1 (in all or in part), or to at least 60% of SEQ ID NO: 1, the remaining portion of the primer being complementary to a sequence flanking SEQ ID NO: 1 in the genome of C. suis.

4. The method of claim 2, wherein the forward and reverse primers comprise a single-strand nucleic acid, with a length between 5 and 50 bases, preferably between 5 and 30 bases, even more preferably between 10-30 bases, or between 15-25 bases.

5. The method of claim 2, wherein the reverse primer is selected from anyone of SEQ ID NOs: 2 to 6, preferably the reverse primer is SEQ ID NO: 2.

6. The method of claim 2, wherein the forward primer is a primer allowing, in combination with the reverse primer, amplification of a C. suis genomic sequence of between 50 400 bp, preferably between 50 and 250 bp, even more preferably 50 and 200 bp.

7. The method of claim 6, wherein the forward primer is selected from anyone of SEQ ID NOs: 7 to 12, preferably the reverse primer is SEQ ID NO: 7.

8. A method for detecting C. suis in a sample, comprising a step of nucleic acid amplification in said sample with a nucleic primer selected from any one of SEQ ID NOs: 2-6.

9. The method of claim 8, comprising a step of nucleic acid amplification in said sample with a pair of a forward and a reverse nucleic primers, the reverse primers being selected from any one of SEQ ID NOs: 2-6 and the forward primer being selected from anyone of SEQ ID NOs: 7-12, preferably the reverse primer SEQ ID NO: 2 and the forward primer is SEQ ID NO: 7.

10. The method of claim 1, wherein amplification is PCR amplification, more preferably qPCR amplification.

11. The method of claim 1, wherein the sample is a biological sample from a mammal, preferably a pig (or piglet).

12. The method of claim 11, wherein the sample is, or is obtained from, faeces or stool, preferably from pig or piglet.

13. The method of claim 12, wherein the sample is or comprises DNA extracted from stool.

14. The method of claim 1, wherein amplification comprises between 5-50 cycles, such as from 20 to 40.

15. The method of claim 10, wherein amplicon is detected with a probe.

16. A method of detecting C. suis comprising using a nucleic primer selected from any one of SEQ ID NOs: 2-6.

17. A method of amplifying C. suis genomic sequence in a sample comprising using a nucleic primer selected from any one of SEQ ID NOs: 2-6.

18. A nucleic acid primer selected from any one of SEQ ID NOs: 2-6.

19. A method of amplifying C. suis genomic sequence in a sample comprising using a pair of nucleic primers, the pair comprising a reverse primer selected from any one of SEQ ID NOs: 2-6 and a forward primer selected from any one of SEQ ID NOs: 7-12.

20. A kit comprising a nucleic acid primer of claim 18 and one or more reagent(s) for performing an amplification.

21. The kit of claim 20, further comprising a primer selected from anyone of SEQ ID NOs: 7-12.

22. The kit of claim 20 or 21, which further comprises container and/or a manual for performing an amplification.
